(19) United States
(12) Patent Application Publication
(10) Pub. No.: US 2024/0201137 A1
(43) Pub. Date: Jun. 20, 2024

(54) MAGNETIC NON-DESTRUCTIVE ANALYSIS AND TESTING FOR ULTRA-HIGH PERFORMANCE CONCRETE

(71) Applicant: University of Florida Research Foundation, Inc., Gainesville, FL (US)

(72) Inventors: Daniel Jesutomi Alabi, Gainesville, FL (US); Christopher Charles Ferraro, Gainesville, FL (US); Joel B. Harley, Gainesville, FL (US); Kyle A. Riding, Gainesville, FL (US)

(21) Appl. No.: 18/566,914

(22) PCT Filed: Jun. 3, 2022
(86) PCT No.: PCT/US2022/032076

§ 371 Date: Dec. 4, 2023

Related U.S. Application Data
(60) Provisional application No. 63/196,478, filed on Jun. 3, 2021.

(51) Int. Cl. G01N 27/72; G01N 33/38

(52) U.S. Cl. CPC G01N 27/72; G01N 33/383

(57) ABSTRACT

Various examples related to magnetic non-destructive analysis and testing of ultra-high performance concrete (UHPC). In one example, a method includes positioning a magnetic sensor on or adjacent to a surface of an ultra-high performance concrete (UHPC) structure; determining, using the magnetic sensor, inductance change of the UHPC structure in two directions that are substantially orthogonal to each other; and determining a fiber orientation within the UHPC structure based upon the determined inductance change in the two directions. In another example, a system includes a support structure that supports at least one magnetic sensor on or adjacent to a surface of a UHPC structure; at least one data analyzer that can determine inductance change of the UHPC structure using the at least one magnetic; and processing circuitry configured to determine a fiber orientation within the UHPC structure based upon the determined inductance change.

### CROSS REFERENCE TO RELATED APPLICATIONS

This application claims priority to, and the benefit of, co-pending U.S. provisional application entitled “Magnetic Non-Destructive Analysis and Testing for Ultra-High Performance Concrete” having Ser. No. 63/196,478, filed Jun. 3, 2021, which is hereby incorporated by reference in its entirety.

### STATEMENT REGARDING SPONSORED RESEARCH OR DEVELOPMENT

This invention was made whole or in part from funding received under Grant No. BDV31 977-105, received from the Florida Department of Transportation.

### BACKGROUND

Ultra-high performance concrete (UHPC) is a versatile and highly durable form of concrete that has a high tensile and flexural strength. Unlike traditional concrete mixture, UHPC is made with extremely fine aggregate and thin steel fibers. Due to this mixture, UHPC achieves much higher strength characteristics than traditional mixes and therefore can be used with less material and lower maintenance. Yet, improperly fabricated ultra-high-performance concrete can become a safety concern if characteristics do not match the design specifications. One of the greatest concerns is the distribution and orientation of steel fibers throughout the concrete mixture. Ideally, fibers should be uniformly distributed and randomly orientated to ensure heterogeneous strength characteristics throughout the concrete member. Yet, how the concrete structure is poured and handled during the setting process can significantly shift the distribution of fibers.

### SUMMARY

Aspects of the present disclosure are related to magnetic non-destructive analysis and testing of ultra-high performance concrete (UHPC). In one aspect, among others, a method comprises positioning a magnetic sensor on or adjacent to a surface of an ultra-high performance concrete (UHPC) structure; determining, using the magnetic sensor, inductance change of the UHPC structure in two directions that are substantially orthogonal to each other; and determining a fiber orientation within the UHPC structure based upon the determined inductance change in the two directions. The method can comprise determining fiber content of the UHPC structure. In one or more aspects, the magnetic sensor can comprise a first coil wound around a first inductor core and a second coil wound around a second inductor core that is substantially orthogonal to the first inductor core.

In various aspects of these embodiments, the magnetic sensor can be excited by a continuous wave at a first frequency to determine the inductance change in the two directions. The magnetic sensor can be excited at a plurality of frequencies to determined corresponding inductance change in the two directions at different frequencies. Fiber content and orientation of the UHPC structure can be determined for each of the different frequencies. The fiber content and orientation can be associated with a depth in the UHPC structure. The fiber orientation can be based upon a ratio of the inductance change in the two directions. In one or more aspects, the magnetic sensor can be supported at a fixed distance away from the surface of the UHPC structure. The magnetic sensor can be supported by a vehicle or carriage configured to allow movement along the surface of the UHPC structure.

In some aspects, the method can comprise adjusting orientation of the magnetic sensor based upon the determined fiber orientation and determining inductance change of the UHPC structure in two directions with the magnetic sensor in the adjusted orientation. The method can comprise repositioning the magnetic sensor to another position along the surface of the UHPC structure and determining inductance change of the UHPC structure in two directions with the magnetic sensor at the other position. The fiber orientation and fiber content can be determined at a plurality of positions along the surface of the UHPC structure. The method can comprise generating a structural image based upon the inductance change at the plurality of positions along the surface of the UHPC structure. The method can comprise determining a location of the magnetic sensor with respect to the UHPC structure when determining the inductance change in two directions.

In another aspect, a system comprises a support structure that supports at least one magnetic sensor on or adjacent to a surface of an ultra-high performance concrete (UHPC) structure; at least one data analyzer in communication with the at least one magnetic sensor, the at least one data analyzer configured to determine inductance change of the UHPC structure using the at least one magnetic sensor, wherein inductance change of the UHPC structure is obtained in two directions that are substantially orthogonal to each other; and processing circuitry configured to determine a fiber orientation within the UHPC structure based upon the determined inductance change in the two directions. In various aspects, the at least one magnetic sensor can comprise first and second magnetic sensors that are substantially orthogonal to each other. The at least one data analyzer can comprises a first data analyzer in communication with the first magnetic sensor, the first data analyzer configured to determine inductance change of the UHPC structure in a first direction; and a second data analyzer in communication with the second magnetic sensor, the second data analyzer configured to determine inductance change of the UHPC structure in a second direction substantially orthogonal to the first direction. The at last one data analyzer can comprise an inductance, capacitance, and resistance (LCR) meter. The processing circuitry can be configured to render measured inductance changes in real-time.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A and 1B illustrate examples of a magnetic sensing system including a magnetic sensor for non-destructive testing and analysis of ultra-high performance concrete (UHPC), in accordance with various embodiments of the present disclosure.

FIGS. 2 and 3 illustrate examples of inductance change from test results of UHPC specimens, in accordance with various embodiments of the present disclosure.

FIG. 4 illustrates the operating principle of the magnetic sensor, in accordance with various embodiments of the present disclosure.

FIG. 5 illustrates an example of inductance change of a magnetic sensor as a factor of height, in accordance with various embodiments of the present disclosure.

FIGS. 6A and 6B are images of an UHPC H-pile used for validation, in accordance with various embodiments of the present disclosure.

FIGS. 7A and 7B are images of a core sample (H3) from the H-pile and CT analysis, in accordance with various embodiments of the present disclosure.

FIG. 8 illustrates an example of self-resonance of a coil or inductor, in accordance with various embodiments of the present disclosure.

FIGS. 9A and 9B illustrate examples of inductance change as a factor of frequency for a UHPC site sample and test slab, in accordance with various embodiments of the present disclosure.

FIG. 10 illustrates an example of a plot of inductance data with respect to positional data, in accordance with various embodiments of the present disclosure.

FIGS. 11A-11D illustrate examples of magnetic sensing systems, in accordance with various embodiments of the present disclosure.

FIG. 12 illustrates an example of dual magnetic sensors, in accordance with various embodiments of the present disclosure.

FIG. 13 illustrates an example of a user interface that can be used with the magnetic sensing systems of FIGS. 11A-11D, in accordance with various embodiments of the present disclosure.

FIGS. 14A and 14B illustrate examples of inductance measurements, in accordance with various embodiments of the present disclosure.

### DETAILED DESCRIPTION

Disclosed herein are various embodiments of methods and systems related to magnetic non-destructive analysis and testing of ultra-high performance concrete (UHPC). Examples of nondestructive systems and methods are presented that can be used to verify the proper construction of the concrete, and in properly fabricated UHPC, can determine the quantity and orientation of fibers. Reference will now be made in detail to the description of the embodiments as illustrated in the drawings, wherein like reference numbers indicate like parts throughout the several views.

Many traditional nondestructive evaluation techniques (ultrasound, electrical methods, etc.) for concrete tend to be far more sensitive to general heterogeneities in the concrete mixture than heterogeneities in fiber content. As a result, these approaches are impractical for this application. However, magnetic strategies can be used to sense the steel fibers with minimal effects from the concrete. That is, the concrete material by itself is not strongly attracted to a magnetic source because most of the materials are paramagnetic (weakly attracted to external magnetic fields). However, the steel fibers that are used to enhance the mechanical performance of UHPC are usually steel, which is a ferromagnetic material, making it sensitive to external magnetic fields.

These steel fibers can be detected by an inductive device whose inductance increases in the presence of a ferromagnetic material. When a system including various coils wrapped around concrete blocks is used, there is a change in the magnetic field around the coils due to the presence of the steel fibers in the concrete block. However, this approach is impractical for field use. It has been shown that a solid framework laid for testing the quantity and the orientation of steel fibers in steel fiber reinforced concrete (SFRC) is capable of detecting steel fiber content in the SFRC and identify fiber orientation in the concrete when the sensor is raised above (i.e., not touching) the specimen. While these results have shown significant promise, the capabilities and limitations of this magnetic method has not been rigorously validated, particularly for field specimens. A system that can measure and map relevant non-destructive information in real-time (such as the 3D spatial distribution of fiber orientations), has not yet been realized.

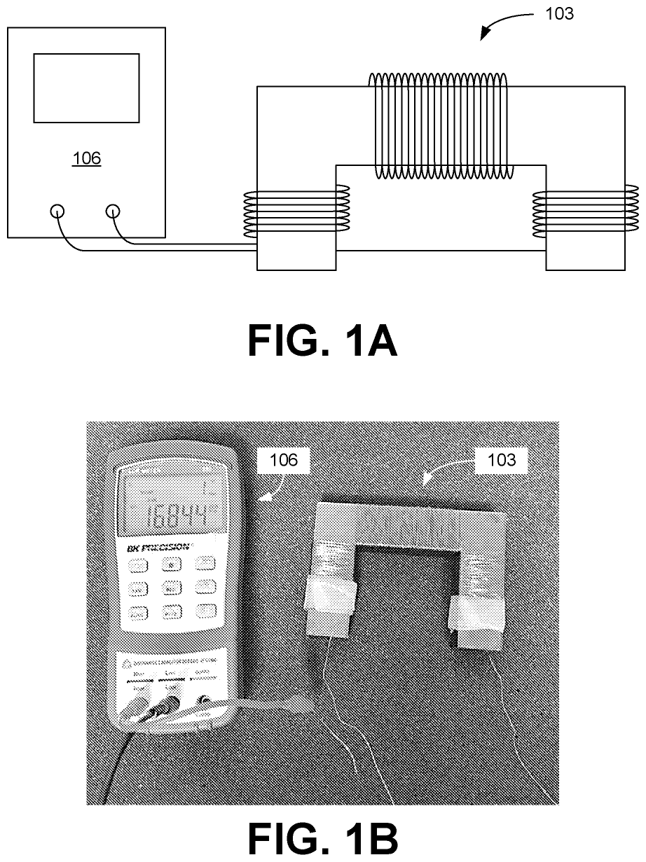

To address these challenges, a magnetic sensor based was designed to detect the fiber volume and orientation of steel fibers in UHPC. FIG. 1A graphically illustrates an example of a system comprising the magnetic sensor 103 and a data analyzer 106 or other appropriate data analysis processing device. The magnetic sensor 103 can comprise an electromagnet having, e.g., an N87 U-shaped ferrite core with a cross-sectional area of, e.g., 28 mm by 20 mm, a height of, e.g., 91 mm, and a length of, e.g., 126 mm was used as the magnetic core of the inductor. Inductor cores usually have low magnetic permeance, which enables better flux flow through them, which helps improve the sensitivity of the device. Coils are formed on the inductor core. For example, a magnetic wire of American Wire Gauge (AWG) 28 was wound around the ferrite core for a total of 320 turns (90 turns on each leg and 140 turns on the length). The number of turns can vary based upon the coil characteristics and magnetic core size. This gave an inductance of about 16.8 mH and 2435 ohms resistance at 1000 Hz, the DC resistance is 3.5 ohms. The ferrite core provides a structure supporting the coils, making it very compact and easy to handle without crushing the coils.

FIG. 1B is an image of the fabricated setup of the experimental magnetic induction sensing device. In the setup of FIG. 1B, the data analyzer 106 was a BK precision 880 LCR meter which can measure changes in magnetic induction in the magnetic sensor 103 when placed in proximity with regions of the UHPC. The data analyzer 106 or other appropriate data analysis processing device can comprise processing circuitry (e.g., including a processor and memory that can execute program applications) configured to analyze measurements obtained using the magnetic sensor 103 and determine, using the measurements, fiber content and/or orientation within the object under test.

Preliminary results show a clear relationship between measured inductance and expected fiber density. To verify this relationship, the sensor system was used detect the volume and orientation of steel fibers in UHPC. The experimental setup included various UHPC samples having fiber percentages ranging from 1 to 3%. The work was further tested using several full-scale UHPC members, including H-piles, I-beams, octagonal piles, and square piles. Results were compared against fiber orientation and content calculated from X-ray computed tomography conducted on cores taken from these members. The non-destructive testing method used is discussed as well as the comparison with CT results.

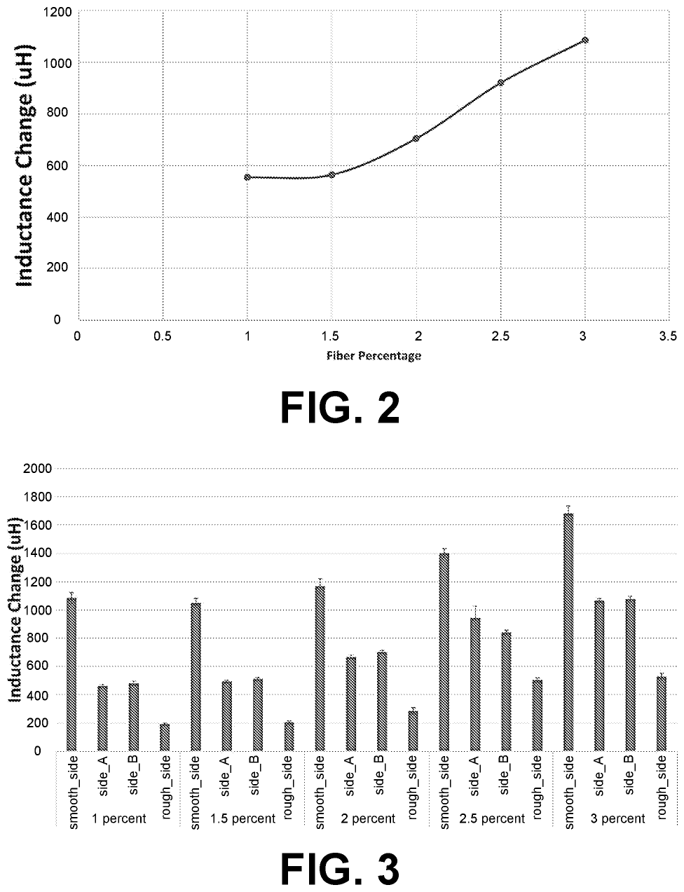

FIG. 2 illustrates an example of the average inductance change (relative to baseline—when the magnetic sensor 103 is not near any ferromagnetic material) when the magnetic sensor 103 is placed near UHPC specimens with varying fiber percentage (by volume). The inductance change should be larger with a corresponding increase in fiber volume in the UHPC as there would be a commensurate increase in the magnetic line of flux linking the sensor which will result in a higher inductance change. It can be shown that the inductance change increases with an increase in the fiber volume in the UHPC using lab specimens that were designed with different fiber percentages ranging from 1% to 3%. Lab UHPC specimens with 1%, 1.5%, 2%, 2.5% and 3% fiber were tested, and their results plotted in FIG. 2. The results of the 1% specimen are similar to the 1.5% specimen. This may be attributed to an issue with the experimental setup as the trend agrees with flexure testing. The result of the fiber percentage was also confirmed by mechanical testing that showed consistency with the NDT method.

FIG. 3 illustrates an example of the inductance change (relative to the baseline—when the magnetic sensor 103 is not near any ferromagnetic material) with the magnetic sensor 103 places near the four sides of a UHPC specimen. The results in FIG. 3 use the same specimens as used in FIG. 2 and shows the effect of settling of the fibers in the UHPC. The smooth side represents the portion of the specimen that was on the bottom of the mold that was used in casting the UHPC specimen. The rough side represents the top of the mold. Sides A and B are just arbitrary sides of the specimen that were on the sides of the mold (i.e., not the top or bottom).

The results show the settling of the fibers, possibly due to the fibers being denser than the other particles in the UHPC. In all of the specimens, the smooth side, which represents the side on the bottom of the mold, has a higher inductance change and therefore a higher fiber content. In contrast, the rough side shows the lowest inductance change and therefore the least amount of fiber content for all the specimens. The other sides show of the specimen shows inductance in-between the top and bottom of the specimen, which further agrees with the settling of the fibers.

Fiber orientation can be measured by rotating the magnetic sensor 103. As the magnetic sensor 103 is rotated the inductance will vary. The measured inductance will be maximum when the fibers are aligned with the magnetic poles. This is because the steel fibers serve as a medium for the magnetic field to propagate through. The ratio of the inductances in when the magnet is oriented at one angle and at a 90-degree difference describes fiber orientation. A ratio of 1 indicates no specific fiber orientation (a mixture of fibers with random orientations) while a ratio greater or less than 1 indicates fiber orientation in one of the two directions.

These preliminary results demonstrate a relationship between fiber density and magnetic inductance. Orientation of the fibers can be identified by rotating the magnetic sensor 103. The magnetic inductance will increase when the magnetic sensor 103 is well-aligned with the fibers. Hence, this technology has the capability to measure fiber density and identify fiber orientation in the UHPC when the sensor is raised above (i.e., not touching) the specimen. The magnetic sensor 103 can be placed into a rolling vehicle or carriage with a rotary encoder (to measure travel distance). The vehicle and magnetic sensor 103 can connect to a data acquisition device (e.g., data analyzer 106 or other appropriate data analysis processing device) to record location and magnetic inductance. The full system can be operated by an inspection team to create nondestructive evaluation images of a concrete structure.

The magnetic sensor 103 can comprise two electromagnets that are perpendicular to each other. This arrangement can enable the device to be able to compare two axes at a time to find the preferential alignment of the fibers. Using vectorial analysis, it can be shown that the higher the inductance change present in either axis, the more preferentially aligned the fibers are towards that axis.

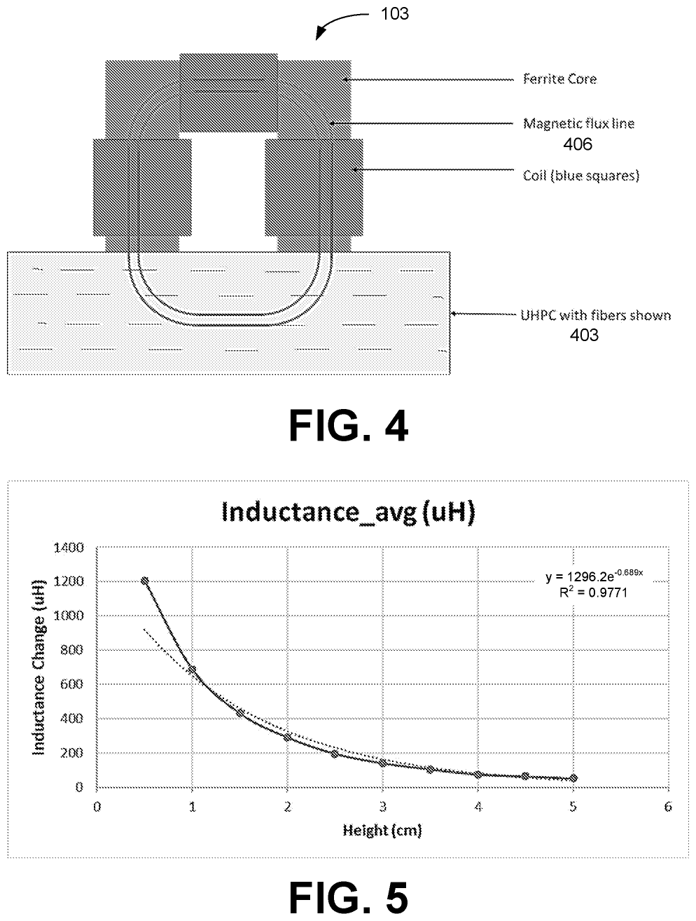

The system uses a method of inductance change to determine both the fiber volume, and the orientation. FIG. 4 illustrates the operating principle of the magnetic sensor 103 showing one of the magnets on a UHPC block 403 with the fibers shown. Lines 406 graphically represents the theoretical flux path. This is the same for both magnets.

The system can comprise, e.g., an N87 U-shaped ferrite core as the inductor core. The ferrite core directs the magnetic flux to provide a focused field without leakage to the environment, which can increase the sensitivity of the sensing device and boost the penetration depth. The core can be, e.g., 126 mm long and can have a cross-sectional area of, e.g., 20 mm by 30 mm. The wire can be AWG 25 with 320 turns. The number of turns increases the sensitivity of the device as more windings will generate more flux, which in turn will improve the sensitivity of the magnetic sensor 103 overall.

FIG. 5 illustrates an example of the sensitivity with distance by showing the inductance change as a factor of height. The magnetic sensor 103 was placed over a steel rebar of about 70 mm in diameter. Measurements were taken at discrete heights and plotted against the corresponding inductance change. A line of best fit shows an exponential decay of the magnetic flux. This data illustrates experimentally how the device will perform in terms of penetration depth and provides calibration of the magnetic sensor 103. This also shows how much or how little a rebar in field specimens will interfere with the readings. As indicated by the plot, the magnetic sensor 103 need not be directly on the surface of the specimen to have usable readings. This result also indicates how much the rebar will interfere with readings based on the amount of actual concrete cover.

### Fiber Analysis and CT Scanning Validation

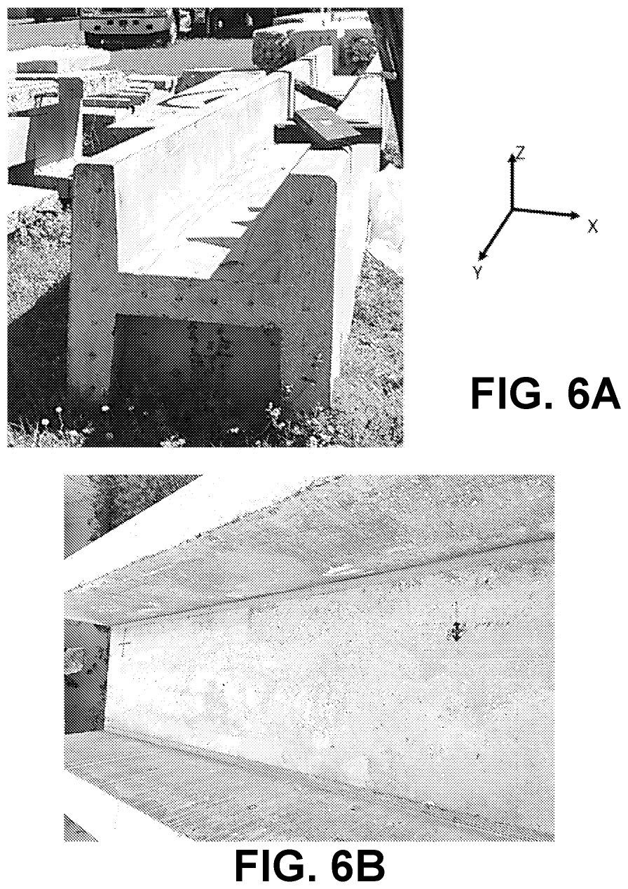

Field F experiments were performed to validate the magnetic sensor readings with a realistic UHPC structure. For this validation, measurements were taken from an UHPC H-pile. FIG. 6A is an image of the H-pile on site. After measurements were taken, a core was removed from the H-Pile for further study, including a CT scan. The core was marked with an orange double-headed arrow to indicate the y-direction and with a black double-headed arrow to indicate the x-direction. FIG. 6B shows the core markings on the H-pile. The y-direction and x-direction will correspond to the parallel and perpendicular directions respectively, of the magnetic device.

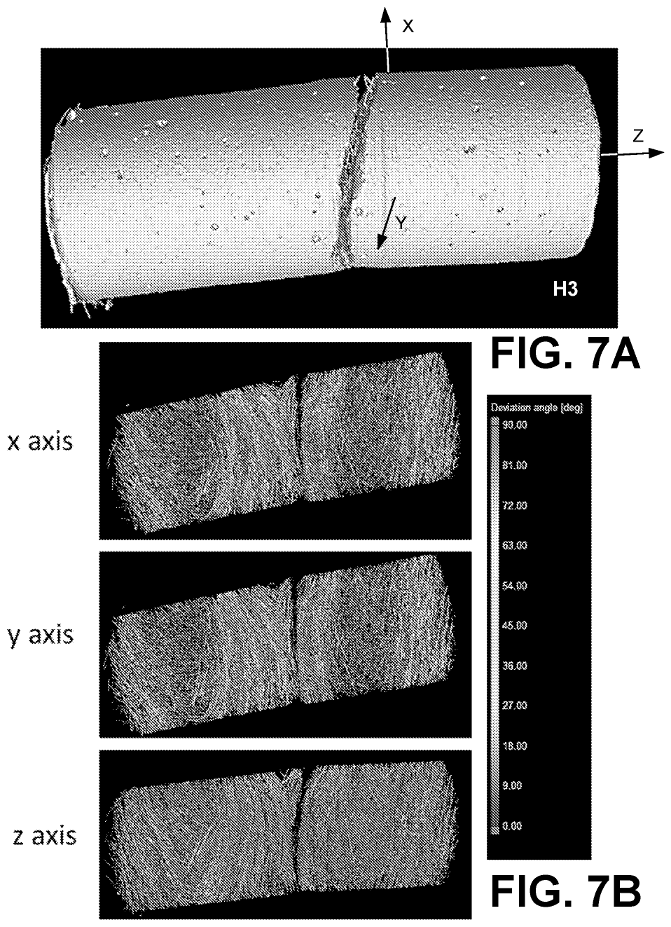

Four cores were taken from this H-pile, all measuring 2-inch diameter and 5-inch height. FIG. 7A shows the third core from the H-pile, which is labeled ‘H3’ for this study. The core H3 was taken at a distance of 20 ft from the end of the H-pile where the measurements began, and 6 inches from right the flange. The core H3 was scanned and oriented as shown in FIG. 7A with the z axis perpendicular to the cored surface and y axis lengthwise for the H pile. FIG. 7B shows the CT image and orientation analysis of core H3. The scans illustrate the deviation angle of the fibers. As seen in the scans, there are distinct layers of concrete that have preferentially oriented fibers. The y-axis view shows more fibers in the y-direction. Very few fibers are oriented in the z-direction, which makes sense because this would be perpendicular to the formed surface.

Quantitative Analysis of CT Scan. Trigonometry can be used to calculate the amount of fiber projected in each direction:

$${\sum }_{θ = 0}^{θ = 90} nX cos ( θ )$$

where n is the amount of fiber with the deviation angle θ. The projection ratios were found to be x/y: 0.59 and y/x: 1.70. This shows that there is roughly 70% more fiber alignment in the y-direction when compared with the x-direction in this location.

Fiber Orientation Analysis from Magnetic Readings. The inductance reading was calculated for the core H3 and the measurement values were ratioed to obtain x/y: 0.81 and y/x: 1.23. This shows that there is roughly 23% more fiber alignment in the y-direction when compared with the x-direction in this location.

The discrepancy between the CT scan and the magnetic reading may be attributed to the reason that the ratio obtained from the CT scan is over the whole volume of the H3 core, which is 5 inches thick. The magnetic method has been shown from the height/depth experiment to be able to scan reliably up to 1 inch, and possible up to 2 inches with some certainty as shown in the height versus inductance change result. The curve illustrating the sensor sensitivity vs. height may be used to better calibrate the magnetic sensor data analysis and obtain agreement between the CT scan and the magnetic measurements.

### Frequency Information

One of the important aspects of the disclose magnetic sensing technology is the frequency. There are two forms of inductance that can be used in practice: direct current (DC) inductance or alternating current (AC) inductance. The type of available excitation, DC or AC, determines the inductance type. The AC inductance is a factor of the alternating current that is driving it. It can be shown that the frequency of the inductor increases with an increase in the frequency of the AC driving it until it reaches a self-resonance where the inductance flips and the inductor starts acting like a capacitor.

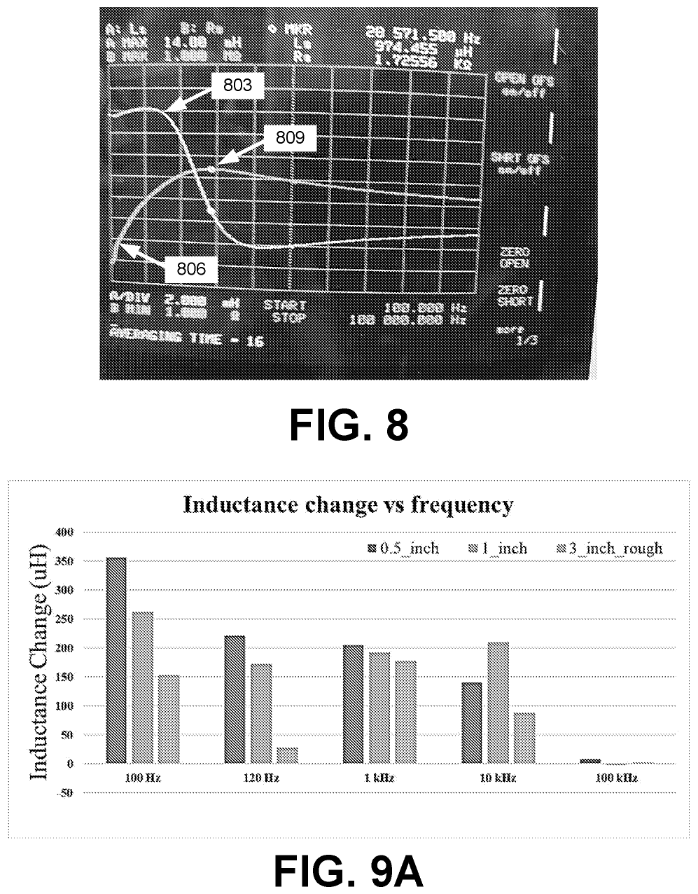

This self-resonance can be explained further by the example shown in FIG. 8, which illustrates the self-resonance of a 1 mH inductor. Curve 803 shows the inductance of the coil and curve 806 shows the resistance of the coil as the frequency is varied. The resonance is the point 809 where resistance is maximum as this gives the worst quality factor. The coil resonates at a frequency of about 32 kHz. Each individual coil has a characteristic self-resonance, so the exemplary graph shown in FIG. 8 will vary based on the characteristics of the individual coil and is just shown as a general representation of what a self-resonance of an inductor would look like.

One of the things that can be inferred from the graph of FIG. 8 is the gradual increase of inductance with increasing frequency up to a point. This can be exploited to improve sensitivity and resolution of the magnetic sensing system. Lower frequencies increase the depth of penetration of the magnetic sensor 103, while higher frequencies increase discrimination or resolution of the magnetic sensor 103. By changing frequencies, the sensing depth in the UHPC can be increased or decreased. This is helpful especially when varying depths of concrete are to be covered, and depths and thickness of buried rebars vary in the UHPC. By varying the frequency, the scan depth can be controlled to scan more depth or less depth of UHPC without much interference from the rebars.

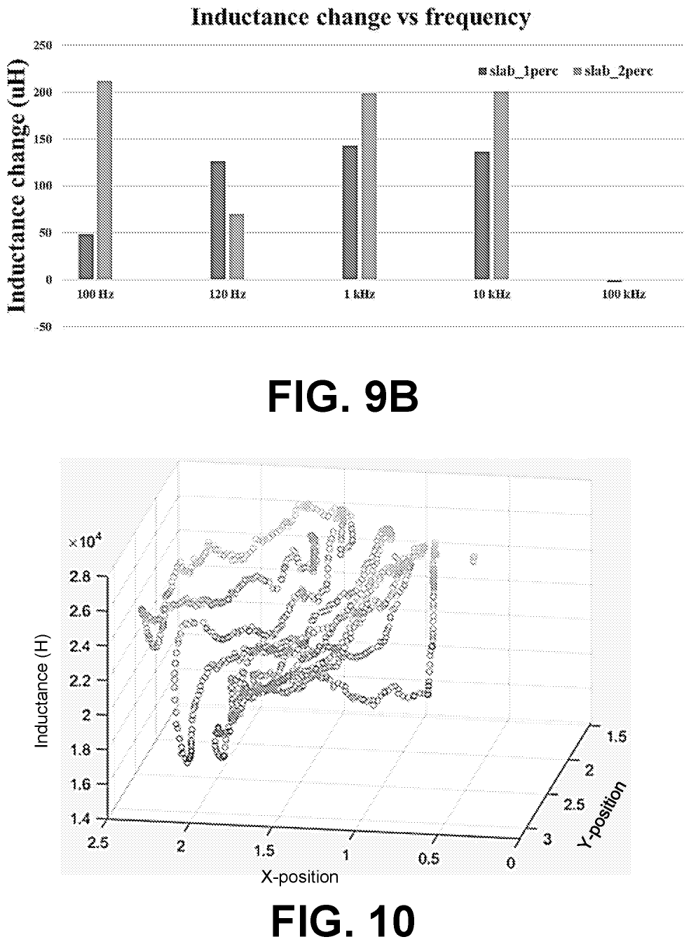

Examples of preliminary test results are illustrated in FIGS. 9A and 9B, which show a strong link between frequency and the inductance change. FIG. 9A illustrates the inductance change versus frequency for a UHPC durability site sample. The specimen contained UHPC with approximately 2% fiber content and three different rebars buried at 0.5, 1, and 3 inches. The specimen had a cross-sectional area of 6 inches by 6 inches, placing the rebar buried at 3 inches at the center of the specimen. FIG. 9B illustrates the inductance change versus frequency for a UHPC test slab. The specimen contains UHPC with approximately 2% fiber content on one side and 1% fiber content on the other side. The 2% fiber was about 2.2 inches-thick and the 1% fiber portion was three fourths of an inch thick. The specimen had a cross-sectional area of 19.5 inches by 17.6 inches.

There is a variation when different frequencies are used to probe the specimens. A frequency of 100 Hz seems to penetrate through specimens better than the other frequencies by the larger inductance change that results. The specimen used for the FIG. 9A result was a pile with a 6-inch by 6-inch cross-section that was about 4 feet long. Three rebars were buried in the specimen to show the influence of rebar on the magnetic results, and by extension, to show the different depths that can be seen and how the rebars influence this result. The specimen sued for FIG. 9B was a 3-inch-thick slab with a cross-sectional area of 19.5 inches by 17.6 inches. The slab contained two layers of UHPCs, each containing different fiber percentages. The first layer contained 2% fibers with a thickness of about 2.2 inches while the second layer contained 1% fibers with a thickness of about 2 0.75 inches.

In FIG. 9A, the labels 0.5_inch, 1_inch, and 3_inch_rough each represent the readings while the magnetic sensor 103 was placed on the sides, with the rebar being 0.5 inches, 1 inch, and 3 inches away from the magnetic sensor 103, respectively. In theory, based on depth, there would be more influence from the 0.5 inches, 1 inch, and 3 inches, in decreasing order. The frequency also plays a role in the value of the inductance change.

In FIG. 9B, slab_1perc and slab_2perc represent the readings while the magnetic sensor 103 was placed on the face with 1 percent fiber mix and 2 percent fiber mix, respectively. In theory, the face with the 2 percent will have a higher reading than the 1 percent face, even before considering the frequency effect. This still holds as can be seen from the readings with varying frequencies. It is possible to penetrate more depth and sense further with lower frequencies than with the higher frequencies.

There are various sources of noise that can be experienced in the implementation of this method. Some of the noise are artificial (manmade) while others are atmospheric. Examples of noise sources include, e.g., electrical mains, TV line frequencies at about 15-16 kHz plus harmonics, modern electronic power supplies (e.g. found in computers), Electric fences and ignition systems, metal detectors, long and medium wave radio transmitters, lightening, long conductors, TV stations, microwave links, mobile phone tower signals, and/or radars, etc. Modern electronic power supplies can produce low-level interfering signals at many tens of kHz but the source needs proximate to the magnetic sensor 103 to be a problem. If one metal detector is transmitting a similar magnetic field to another, they are likely to interfere with each other if close. Long and medium wave radio transmitters are not a big problem, unless the magnetic sensor 103 is close to the transmitter.

A more significant noise source for the magnetic sensor 103 is the electrical mains. Since the US mains supply is at a frequency of 60 Hz, they can produce interference at integer multiples of this frequency. For example, 120 Hz, 180 Hz, 240 Hz, etc., are very good sources of interference. Looking at the results in FIGS. 9A and 9B, there is possible interference of the 120 Hz results which would help explain the results discrepancy. Appropriate shielding of the system may mitigate some of these effects.

The analysis data may be used to generate structural images or plots. FIG. 10 is an example that incorporates inductance data with 2D positional data. The positional data can be computed using, e.g., a commercial ultrasonic positioning system. The image shown in FIG. 10 represents a flat surface area with an aluminum plate in the center. The inductance measured by the magnetic sensor 103 decreases in the region around the aluminum plate. The x and y axes are spatial dimensions measured in meters, and the z axis is the inductance measurement. The plate can be seen as an area of low inductance in the center of the plotted region. This example also shows some anomalies, like the region of extremely low inductance off the lower two edges of the aluminum plate, which have since been removed from the data collection process through further testing.

Various examples of methods and systems related to magnetic non-destructive analysis and testing of UHPC have been presented. The methodology uses a continuous wave with multiple frequencies rather than a pulse that varies over time. Using a continuous signal enables the system to detect homogeneous volume of the fibers in the UHPC as well as the orientation of those fibers. The use of two different sensors in an orthogonal (or substantially orthogonal) configuration can improve sensing of the fiber orientations.

### Sensing System Designs

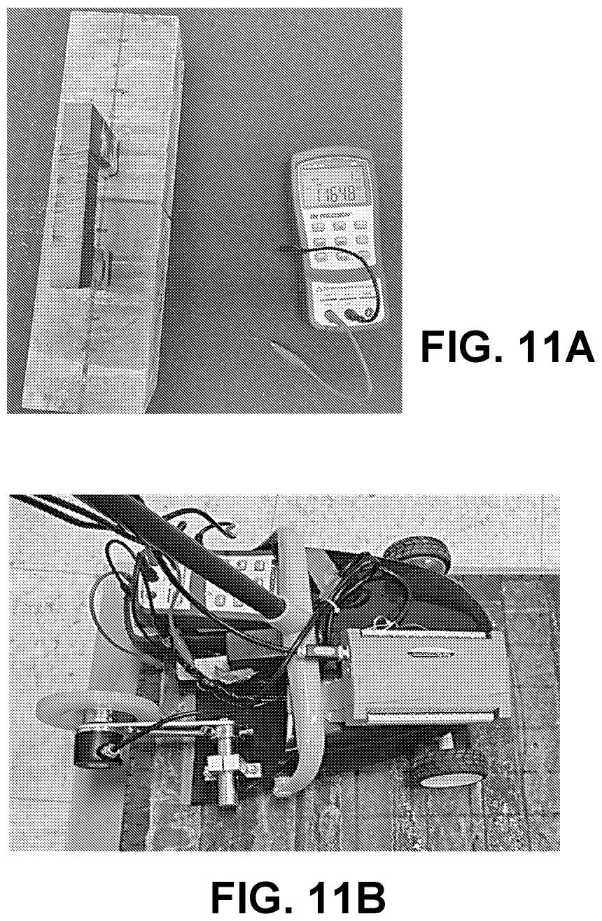

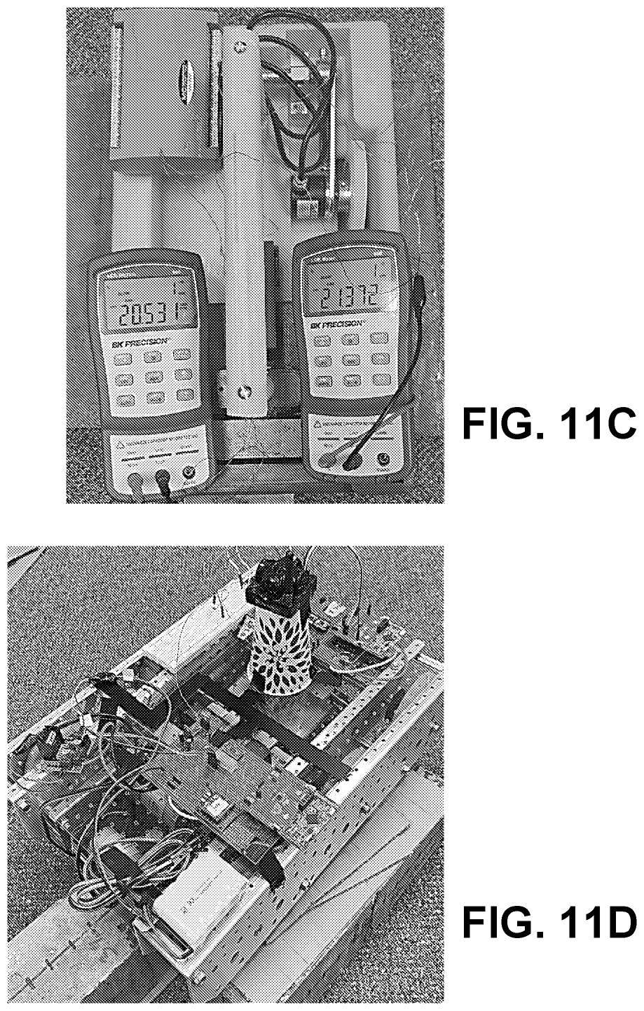

A range of prototypes for non-destructive testing and analysis of UHPC have been fabricated using a magnetic sensing system. FIGS. 11A-11D illustrate examples of magnetic sensing systems including a single sensor system, a dual sensor system, an enclosed sensor system, and an automated system.

Single Sensor System. FIG. 11A is an image showing the magnetic sensing system of FIGS. 1A and 1B including a data analyzer 106 (e.g., an LCR meter) with a magnetic sensor 103 (e.g., comprising electromagnets) positioned over a UHPC member. The initial system is a single sensor system comprising one magnet measuring, e.g., 96 mm in length, 28 by 30 mm in cross-sectional area, and wound with AWG 25 wire individually. The sensor system also comprises processing circuitry and other components. The magnet is not self-exciting but is connected to an excitation circuit. In the example of FIG. 11A, a BK Precision 880 Dual-Display Handheld inductance, capacitance, and resistance (LCR) meter was used as both the exciter and the reader. The LCR meter sends about 50 mA of current through the magnet coils to create a magnetic field. When the magnetic field interacts with the steel fibers, the interaction changes the magnetic field and consequently changes the inductance read by the LCR meter and stored on, e.g., a computer, tablet, smartphone or other appropriate storage device.

As shown in FIG. 11A, the electromagnet was connected to the LCR meter and placed directly on the test specimen and multiple readings were taken and recorded, e.g., by connecting the LCR meter to a computer or other computing device or processing circuitry for direct acquisition with the accompanying software. The procedure can include the following operations. First, the LCR meter is turned on, and the ‘PRI/AUTO’ button on the bottom-left corner of the LCR meter is pressed until the display shows ‘L’ on the top-left corner of the display. The ‘L’ stands for inductance reading, and ‘C’ stands for the capacitance measurement that is displayed on the screen when the LCR meter is first turned on. The frequency of choice can be selected by pressing the ‘FREQ/REC’ button at the middle of the buttons, until the desired frequency is shown on the display. The frequency choices on this particular LCR meter are 100, 120, 1000, 10 k, and 100 k Hz.

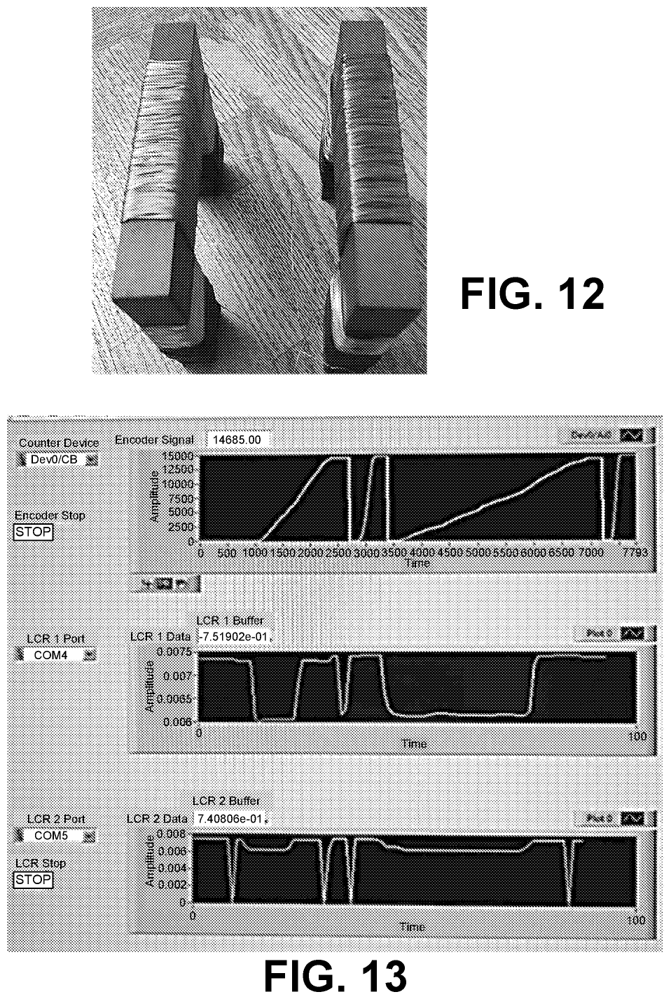

Dual Sensor System. FIG. 11B is an image showing a magnetic sensing system comprising two electromagnets, LCR meters, an encoder for location measurement, and data acquisition system all positioned on a wheeled support structure. This two-sensor system comprises two magnets measuring, e.g., 96 mm in length, 28 by 30 mm in cross-sectional area, with 210 turns of AWG 25 individually connected to an LCR meter each. Two wheels, an encoder, and data acquisition system are all mounted on the wheeled support structure, which includes a long handle to facilitate movement. The system can be directly connected to a Dell workstation or other computing device or processing circuitry. An image of the fabricated dual magnet sensors is shown in FIG. 12.

The magnetic sensing system of FIG. 11B includes an encoder and a data acquisition system. The encoder enables accurate collection of data relating to the position of the magnet sensors with time. The encoder can be connected to a wheel that revolves as the magnetic sensing system moves, and the data acquisition system can collect the encoder data while logging it onto the computer or other computing device or processing circuitry. This can indicate where each data point is collected to make an accurate log of the position as the inductance change is observed. The term inductance change is used to describe the difference in the inductance measured in air and that measured on a UHPC prism or specimen. This can be used to determine how much ferromagnetic steel fibers are present.

After the magnetic sensing system is turned on and the LCR meters are set as previously described, the software is initiated on the computer or other computing device or processing circuitry and run while the magnetic sensing system is being held in air, to get baseline measurements. The magnetic sensing system can then be placed on the surface of the specimen (which has been cleared of debris), making sure the wheels and the encoder can move as smoothly as possible on the specimen's surface. The magnetic sensing system is then slowly moved along the surface to be scanned while wheeling the magnetic sensing system with the handle as shown in FIG. 11B. The magnetic sensing system collects inductance data, and the encoder collects the positional data allowing mapping of the data to a position. After the measurement is completed, measurements can be repeated on the same specimen, or the magnetic sensing system can be moved on to another specimen and the same process repeated.

Enclosed Dual Sensor System. FIG. 11C is an image showing a magnetic sensing system similar to the dual sensor system of FIG. 11B. In this version, the number of turns for the dual magnet sensors was increased to 400 turns of the same AWG 25 wire. The wheeled support structure was reconfigured as an enclosure with 4 wheels to make the magnetic sensing system more compact and easier to handle. The enclosure was printed using polylactide (PLA) with a dense fill to make it as sturdy as possible. The 3D printed enclosure measured 16 in.×10 in.×6 in. The wheels can be mounted to the enclosure, and a slot to receive the encoder can be provided in the enclosure to make it more compact. A wooden handle was used, rather than a 3D printed plastic handle, to provide adequate support for the weight of all the electromagnets, encoder, and the data acquisition system.

The enclosed dual sensor system operates much like the dual sensor system of FIG. 11B. The difference in operation is the enclosed dual sensor system is pushed using the wooden handle on the 3D printed enclosure rather than the long handle of FIG. 11B. In addition, this design has the advantage of being easier to pick up and easier to scan a vertical wall than with the wheeled support version. Both inductance readings from the two LCR meters and encoder data are collected as the concrete member is scanned.

Automated Sensor System. FIG. 11D is an image of an automated magnetic sensing system comprising a robot system with two electromagnets inside to calculate the inductance change of a specimen. The automated magnetic sensing system also comprises a mapping system that uses a positioning system that can be calibrated to calculate the position of the sensors at all times and then map this position to the inductance change calculated. The automated magnetic sensing system can include a support structure arranged to enable it to run remotely on many different sizes of concrete. To accomplish this, a chassis can be chosen and/or modified to adjust or minimize the width.

The Andymark Configurable TileRunner chassis was chosen because it can be configured to meet the application needs. The motor included in the system was a NeveRest 60 with a 9:7 gear ratio. The Neverest 60 has a free speed of 105 rpm and with the 9:7 gear ratio the rpm drops to 135 rpm; with the wheel diameter of 4 in, the drive train speed under load is 1.91 fps (0.58 m/s). To further decrease the speed, a 6V battery was used on the 12V DC motors, dropping the speed to 0.955 fps (0.29 m/s). In the center of the chassis, a box for housing the detection circuit was installed such that it can be easily removed for analyzing surfaces smaller than the width of the chassis. This allows for a more flexible testing apparatus. The box was 3-D printed with drop-down slots for the detection coils on the bottom so the coils can get as close to the concrete as possible. A perforated polycarbonate sheet was mounted on the top so the electronics and positioning equipment can be secured. To access the detection coils, the top can be removed and the coils taken out.

This automated magnetic sensing system was controlled by a Launchpad microprocessor. For example, the microprocessor can communicate with an induction-to-digital converter integrated circuit (IC), which is connected directly to a detection coil circuit. Measurements can be returned to the microprocessor at regular timer intervals. The microprocessor can be configured to simultaneously receive data requests from a host computer and respond with packets of induction data at the relevant time intervals. The central beacon of the positioning system of the automated magnetic sensing system, which is mounted on the vehicle, can collect real time positional data and use the microprocessor to relay the positional data to the host computer. The vehicle can be designed to drive at a consistent speed and take measurements at regular intervals. This results in a relatively even distribution of data points over the scanned area. The robotic system can interface with, e.g., MATLAB code written for the acquisition and processing of the acquired data. It can also display a 3D plot representation of the position in x and y coordinates. The inductance values can be represented by a color gradient of their magnitudes in the z coordinate of the 3D plot. The raw data can also be stored for later access and use.

User Interface. A user interface was designed and implemented in LabVIEW, a graphical programming environment made by National Instruments (NI). The backend comprises the acquisition and data saving loops for the encoder and the inductance, capacitance, and resistance (LCR) devices. The data acquisition rate, sampling rate, and buffer size can be set in the software backend to ensure collection and storage of as much information as the sensors are able to acquire per second. FIG. 13 illustrates an example of a frontend comprising a control interface to start and stop the acquisition of data. It also includes chart plotters to visualize the acquired data in real time. The example of the interface of FIG. 13 includes three waveform charts that allows the data to be displayed in real time. This enables visualization of the data, allowing decisions to be make as needed during scanning.

FIG. 13 shows a LabVIEW interface that was implemented with software. The interface includes three waveform charts that allow a user to look at the data in real-time. The user interface can be configured to allow the naming of the files for storing. Different file names can be used for the encoder, and the first and second LCRs. In addition, the user interface can include individual boxes that display error if there is one, and can give a brief description of the error. The user interface can allow for user input, such as a run button (e.g., in the shape of an arrow) that can be used to run the application, and a stop button that can be used to stop data acquisition when the scan is done. There can also be stop buttons that can be used to stop either the encoder loop or the LCR loop in case there is a need.

### System Reliability and Robustness Assessment

A system reliability and robustness assessment was performed by comparing the results of repeated scans to see how repeatable the measurements are. The results of the dual sensor system of FIG. 11B were compared against the results of the dual sensor system of FIG. 11C to see if updating the sensor gave a repeatable result, bearing in mind the limitations of human involvement in the data collection, and that the amount of time that passed between repeated measurements can also contribute to the error.

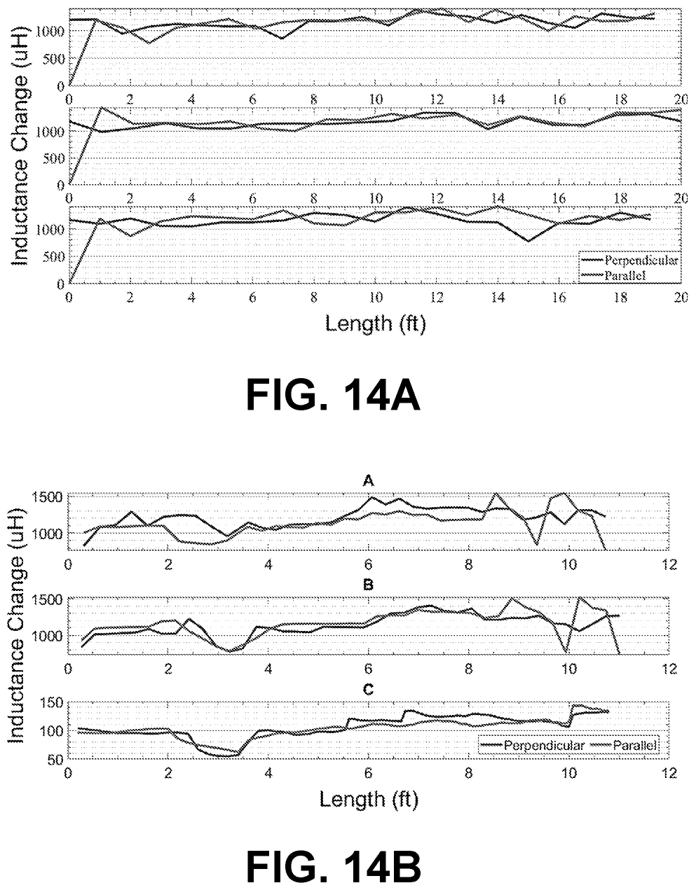

Measurement Repeatability. For the different field specimens we scanned, we decided to take repeated measurements to see how repeatable our measurements are, while bearing in mind that we might have a lot of errors from the operator, the paths we took etc., FIG. 14A illustrates examples of the repeated measurements taken over the Box Beam using the dual sensor system of FIG. 11C. FIG. 14A shows 3 repeated measurements over the same path on the Box Beam. The measurements were each taken and repeated while trying to follow the same path as closely as possible. The similarities and differences in the measurements are visible, but overall, it is possible to see the consistencies. The consistency between the results shows a very good repeatability. The observed differences may be attributed to a number of reasons, from difference in acquisition paths, to overall noise in the system.

Comparison of Magnetic Sensing Systems. Data was collected over different days using the dual sensor systems of FIGS. 11B and 11C in the field, and compared with each other to determine if there are similar trends while using the different versions of the sensor system on the same specimen. FIG. 14B shows a comparison between the data collected over an octagonal pile with the form liner pattern using the dual sensor systems of FIGS. 11B and 11C. Note here that the data collected using the dual sensor system of FIG. 11C was collected after two (2) cores were taken from the pile. The measurements of (A) and (B) were taken using the dual sensor system of FIG. 11C, while the measurements of (C) were taken using the dual sensor system of FIG. 11B. In the measurements of (A) and (B), cores had been taken from the member, one around the 9 ft. mark, and the other around the 11 ft. mark, accounting for the sharp dips around these points. The sharp increase around the 10 ft. mark for the measurements of (C) is due to the absence of the form liner pattern. From FIG. 14B, it can be seen that there is repeatability of results using both the dual sensor systems of FIGS. 11B and 11C. The main difference is the 10 times gain in the inductance change from the dual sensor systems of FIGS. 11B and 11C which can help provide a better inductance change resolution.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A method, comprising:
positioning a magnetic sensor on or adjacent to a surface of an ultra-high performance concrete (UHPC) structure;
determining, using the magnetic sensor, inductance change of the UHPC structure in two directions that are substantially orthogonal to each other; and
determining a fiber orientation within the UHPC structure based upon the determined inductance change in the two directions.

2. The method of claim 1, comprising determining fiber content of the UHPC structure.

3. The method of claim 1, wherein the magnetic sensor comprises a first coil wound around a first inductor core and a second coil wound around a second inductor core that is substantially orthogonal to the first inductor core.

4. The method of claim 1, wherein the magnetic sensor is excited by a continuous wave at a first frequency to determine the inductance change in the two directions.

5. The method of claim 4, wherein the magnetic sensor is excited at a plurality of frequencies to determined corresponding inductance change in the two directions at different frequencies.

6. The method of claim 5, wherein fiber content and orientation of the UHPC structure are determined for each of the different frequencies.

7. The method of claim 6, wherein the fiber content and orientation is associated with a depth in the UHPC structure.

8. The method of claim 1, wherein the fiber orientation is based upon a ratio of the inductance change in the two directions.

9. The method of claim 1, comprising adjusting orientation of the magnetic sensor based upon the determined fiber orientation and determining inductance change of the UHPC structure in two directions with the magnetic sensor in the adjusted orientation.

10. The method of 1, comprising repositioning the magnetic sensor to another position along the surface of the UHPC structure and determining inductance change of the UHPC structure in two directions with the magnetic sensor at the other position.

11. The method of claim 1, wherein the fiber orientation and fiber content are determined at a plurality of positions along the surface of the UHPC structure.

12. The method of claim 11, comprising generating a structural image based upon the inductance change at the plurality of positions along the surface of the UHPC structure.

13. The method of claim 1, wherein the magnetic sensor is supported at a fixed distance away from the surface of the UHPC structure.

14. The method of claim 12, wherein the magnetic sensor is supported by a vehicle or carriage configured to allow movement along the surface of the UHPC structure.

15. The method of claim 1, comprising determining a location of the magnetic sensor with respect to the UHPC structure when determining the inductance change in two directions.

16. A system, comprising:
a support structure that supports at least one magnetic sensor on or adjacent to a surface of an ultra-high performance concrete (UHPC) structure;
at least one data analyzer in communication with the at least one magnetic sensor, the at least one data analyzer configured to determine inductance change of the UHPC structure using the at least one magnetic sensor, wherein inductance change of the UHPC structure is obtained in two directions that are substantially orthogonal to each other; and
processing circuitry configured to determine a fiber orientation within the UHPC structure based upon the determined inductance change in the two directions.

17. The system of claim 16, wherein the at least one magnetic sensor comprises first and second magnetic sensors that are substantially orthogonal to each other.

18. The system of claim 17, wherein the at least one data analyzer comprises:
a first data analyzer in communication with the first magnetic sensor, the first data analyzer configured to determine inductance change of the UHPC structure in a first direction; and
a second data analyzer in communication with the second magnetic sensor, the second data analyzer configured to determine inductance change of the UHPC structure in a second direction substantially orthogonal to the first direction.

19. The system of claim 16, wherein the at last one data analyzer comprises an inductance, capacitance, and resistance (LCR) meter.

20. The system of claim 16, wherein the processing circuitry is configured to render measured inductance changes in real-time.
